(19) United States
(12) Patent Application Publication
(10) Pub. No.: US 2011/0177064 A1
(43) Pub. Date: Jul. 21, 2011

(54) Compositions and Methods for Treatment of Ovarian Cancer

(75) Inventors: Kathleen R. WHITEMAN, Wilmington, MA (US); James J. O'Leary, Newton, MA (US); Robert John Lutz, Wayland, MA (US)

(73) Assignee: ImmunoGen, Inc., Waltham, MA (US)

(21) Appl. No.: 13/011,664

(22) Filed: Jan. 21, 2011

Related U.S. Application Data
(60) Provisional application No. 61/297,188, filed on Jan. 21, 2010.

(51) Int. Cl. A61K 39/395; A61P 35/00

(52) U.S. Cl. 424/133.1

(57) ABSTRACT

The present invention relates to surprisingly effective anti-cancer drug combinations, pharmaceutical compositions comprising the same, and uses thereof in the treatment of ovarian cancer. In particular, the present invention is based on the discovery that the administration of a CD56 antibody linked to a cytotoxic compound (e.g.,, an immunoconjugate) in combination with at least two chemotherapeutic agents (in particular a taxane compound and a platinum compound), improves the therapeutic index in the treatment of ovarian cancer over and above the additive effects of the anticancer agents used alone. In one embodiment of the invention, combinations of the CD56 antibody, or fragment thereof, linked to a cytotoxic compound plus additional chemotherapeutic agents have a synergistic effect in the ovarian cancer therapeutic index. The present invention also provides methods of modulating the growth of selected cell populations, such as ovarian cancer cells, by administering a therapeutically effective amount of such combinations.

### CROSS-REFERENCE TO RELATED APPLICATIONS

This application claims benefit under 35 U.S.C. §119(e) of U.S. Provisional Patent Application No. 61/297,188 filed on Jan. 21, 2010, which is hereby incorporated by reference in its entirety herein.

### BACKGROUND OF THE INVENTION

Ovarian cancer is the most common cancer of the female reproductive tract, presenting an estimated 22,430 new cases and 15,280 deaths in the United States in 2007 (Jemal et al., CA Cancer J. Clin. 2007, 57(1):43-56). Approximately 70% of ovarian cancers are diagnosed at advanced stage and only 30% of women with such cancers can expect to survive 5 years (Cho and Shih, Annu. Rev. Pathol. 2009, 4:284-313).

Current treatments for ovarian cancer include surgery, radiation therapy, chemotherapy, and combinations thereof. The standard first-line chemotherapy for ovarian cancer is a combination of a taxane and a platinum-containing drug. However, such combinations present toxicity risks for patients, and resistance to cytotoxic chemotherapy is the main cause of therapeutic failure and death in women suffering from ovarian carcinoma. See, e.g., Lage and Denkert, Recent Results Cancer Res. 2007, 176:51-60. Furthermore, advanced ovarian cancer treatment with a platinum agent in combination with a taxane is currently limited by a 5-year survival rate of approximately 45%. See, e.g., March et al, Journal of Clinical Oncology, 2007, 25(29):4528-4535.

Xenograft models, e.g., where ovarian cancer cells have been injected either subcutaneously or into the peritoneal cavity, have been used extensively for the testing of novel therapeutics or modified regimens for administration of standard chemotherapeutic drugs. See, e.g., Vanderhyden et al., Reproductive Biology and Endocrinology, 2003, 1:67.

Anti-cancer drugs with different mechanisms of killing, e.g., having different targets in the cell, have been used in combination. For example, combinations of a maytansinoid immunoconjugate comprising a maytansinoid compound (e.g., DM1) linked to a monoclonal antibody (e.g., an anti-CD56 antibody) and (1) paclitaxel, (2) cisplatin and etoposide, (3) docetaxel were used in the small cell lung cancer (SCLC) xenograph model as disclosed in U.S. Pat. Nos. 7,303,749 and 7,601,354, which are incorporated herein by reference in their entirety. In addition, combinations of a maytansinoid immunoconjugate comprising a maytansinoid compound linked to a monoclonal antibody and (1) a proteasome inhibitor (bortezomib), (2) an immunomodulatory agent/anti-angiogenic agent (thalidomide or lenalidomide), or (3) a DNA alkylating agent (melphalan), with the optional further addition of a corticosteroid (dexamethasone) were used in the multiple myeloma xenograph model.

In experimental systems where anti-cancer drugs with different mechanisms of killing are combined, it has been observed that the anti-cancer drugs with independent targets (mutually exclusive drugs) either behave in an additive, synergistic, or antagonistic manner. Chou and Talalay developed a mathematical method to accurately describe such experimental results in a qualitative and quantitative manner (Chou and Talalay, Adv. Enzyme Regul. 1984, 22:27-55). Chou and Talalay showed that a combination of two mutually exclusive drugs will show the same type of effect over the whole concentration range, that is, the combination will show an additive, a synergistic, or an antagonistic type of effect. Most drug combinations show an additive effect. In some instances, however, the combination shows less or more than an additive effect. These combinations are called antagonistic or synergistic, respectively. Antagonistic or synergistic effects are generally considered unpredictable, and are unexpected experimental findings. See Knight et al., BMC Cancer 2004, 4:83; T. H. Corbett et al., Cancer Treatment Reports, 1982, 66:1187; and Tallarida, J. Pharmacol. Exp. Ther., 2001 298(3):865-72.

There is a need in the art for new and more effective methods for treating ovarian cancer. Furthermore, there is still a need for finding drug combinations that show synergism and can be effectively used for the treatment and prevention of cancer, e.g., ovarian cancer. The present invention is directed to such methods and drug combinations.

### BRIEF SUMMARY OF THE INVENTION

The present invention relates to anti-cancer combinations, pharmaceutical compositions comprising the same, and the use thereof in the treatment of ovarian cancer. In particular, the present invention is based on the discovery that the administration of an antibody that specifically binds CD56 linked to a cytotoxic compound (e.g., an immunoconjugate) in combination with at least two chemotherapeutic agents (in particular a taxane compound (such as paclitaxel or docetaxel)) and a platinum compound (such as a carboplatin, a cisplatin, an oxaliplatin, an iproplatin, an ormaplatin, or a tetraplatin compound), improves the therapeutic index in the treatment of ovarian cancer over and above the additive effects of the anticancer agents used alone in a mouse/human (xenograft) model system. In one embodiment of the invention, combinations of an antibody that specifically binds CD56 linked to a cytotoxic compound (i.e., an “immunoconjugate”) plus additional chemotherapeutic agents have a synergistic effect in the ovarian cancer therapeutic index (compared to expected combined additive effects of the single compounds and agents alone). The present invention also provides methods of modulating the growth of selected cell populations, such as ovarian cancer cells, by administering a therapeutically effective amount of such combinations.

In one embodiment, pharmaceutical compositions of the invention comprise a humanized antibody N901-maytansinoid conjugate (huN901-DM1 or IMGN901), a taxane compound, and a platinum compound. In one embodiment the taxane compound in the pharmaceutical composition is one or both of paclitaxel or docetaxel. In one embodiment the platinum compound in the pharmaceutical composition is one or any combination of two or more of a carboplatin, a cisplatin, an oxaliplatin, an iproplatin, an ormaplatin, or a tetraplatin compound. In one embodiment, pharmaceutical compositions of the invention further comprise a pharmaceutically acceptable carrier.

In one embodiment, the immunoconjugate is a humanized antibody N901-maytansinoid conjugate (huN901-DM1 or IMGN901) administered in combination with a taxane compound and a platinum compound, wherein the combination has therapeutic synergy or improves the therapeutic index in the treatment of ovarian cancer compared to the additive effects of using the immunoconjugate alone, the taxane compound alone, the platinum compound alone (or any combination of the preceding two in the absence of the third). In one embodiment the taxane compound is one or both of paclitaxel or docetaxel. In one embodiment the platinum compound is one or any combination of two or more of a carboplatin, a cisplatin, an oxaliplatin, an iproplatin, an ormaplatin, or a tetraplatin compound.

“Therapeutic synergy,” as used herein, means that a combination of a conjugate and one or more chemotherapeutic agent(s) produce a therapeutic effect in ovarian cancer treatment which is greater than the additive effects of a conjugate and chemotherapeutic agents when each are used alone.

These and other aspects of the present invention are described in detail herein.

### BRIEF DESCRIPTION OF THE DRAWINGS/FIGURES

FIG. 1: Shows the anti-tumor effect of IMGN901 treatment only (at two different doses versus control) in OVCAR-3 human ovarian carcinoma xenografts.

FIG. 2: Shows the anti-tumor effect of combination therapy of COLO 720E human ovarian carcinoma xenografts using IMGN901 and paclitaxel plus carboplatin at two different doses versus IMGN901 and paclitaxel plus carboplatin alone (at two different doses).

FIG. 3: Shows the anti-tumor effect of reduced doses of IMGN901 and paclitaxel plus carboplatin (i.e., “low-dose” combination therapy) in established subcutaneous COLO 720E human ovarian carcinoma xenografts.

### DETAILED DESCRIPTION OF THE INVENTION

### Ovarian Cancer.

Ovarian cancer is a cancerous growth arising from different parts of the ovary. The most common form of ovarian cancer (≧80%) arises from the outer lining (epithelium) of the ovary. However, the Fallopian tube (epithelium) is also prone to develop into the same kind of cancer as seen in the ovaries. Since the ovaries and tubes are closely related to each other, it is hypothesized that these cells can mimic ovarian cancer. Other forms of ovarian cancer can arise from egg cells (i.e., a germ cell tumor). The risk of ovarian cancer increases with age and decreases with pregnancy. Lifetime risk has been estimated at about 1.6%, but women with affected first-degree relatives have a higher (˜5%) risk. Women with a mutated BRCA1 or BRCA2 gene carry a risk between 25% and 60% depending on the specific mutation. Ovarian cancer is the fifth leading cause of death from cancer in women and the leading cause of death from gynecological cancer.

The present invention provides improved pharmaceutical compositions and methods for use in the treatment of ovarian cancer.

### Conjugates and Immunoconjugates

One component of the present invention utilizes and a CD56 antibody linked or “conjugated” to a cytotoxic compound (e.g., a maytansinoid compound such as DM1 (described further below)) to produce a “conjugate.” Thus, when the CD56 antibody (or an antigen-binding fragment thereof; such as a fragment containing the antigen-binding domain of a CD56 antibody) is linked to a cytotoxic compound, this combined antibody/cytotoxic compound moiety is referred to herein as an “immunoconjugate.” Immunoconjugates of the present invention are combined with additional cytotoxic compounds or chemotherapeutic agents to produce synergistic effects (synergy) useful in the treatment of ovarian cancer.

### Synergy

Chou and Talalay (Adv. Enzyme Regul., 22:27-55 (1984)) developed a mathematical method to describe the experimental findings of combined drug effects in a qualitative and quantitative manner. For mutually exclusive drugs, they showed that the generalized isobol equation applies for any degree of effect (see page 52 in Chou and Talalay). An isobol or isobologram is the graphic representation of all dose combinations of two drugs that have the same degree of effect, for example combinations of two cytotoxic drugs will affect the same degree of cell kill, such as 20% or 50% of cell kill. In isobolograms, a straight line indicates additive effects, a concave curve (curve below the straight line) represents synergistic effects, and a convex curve (curve above the straight line) represents antagonistic effects. These curves also show that a combination of two mutually exclusive drugs will show the same type of effect over the whole concentration range, either the combination is additive, synergistic, or antagonistic. Most drug combinations show an additive effect. In some instances however, the combinations show less or more than an additive effect. These combinations are called antagonistic or synergistic, respectively. Antagonistic or synergistic effects are unpredictable, and are unexpected experimental findings. A combination manifests therapeutic synergy if it is therapeutically superior to one or other of the constituents used at its optimum dose. See, T. H. Corbett et al., Cancer Treatment Reports, 66, 1187 (1982). Tallarida R J (J Pharmacol Exp Ther. 2001 September; 298 (3):865-72) also notes “Two drugs that produce overtly similar effects will sometimes produce exaggerated or diminished effects when used concurrently. A quantitative assessment is necessary to distinguish these cases from simply additive action.”

A synergistic effect may be measured using the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. In another embodiment, a synergistic effect is determined by using the fractional inhibitory concentration (FIC). This fractional value is determined by expressing the IC50 of a drug acting in combination, as a function of the IC50 of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

That the unpredictability of antagonistic or synergistic effects is well known to one of skill in the art is demonstrated in several other studies, such as, by Knight et al. See, BMC Cancer 2004, 4:83. In this study, the authors measured the activity of gefitinib (also known as Iressa) alone or in combination with different cytotoxic drugs (cisplatin, gemcitabine, oxaliplatin and treosulfan) against a variety of solid tumors including breast, colorectal, esophageal and ovarian cancer, carcinoma of unknown primary site, cutaneous and uveal melanoma, non-small cell lung cancer (NSCLC) and sarcoma.

They discovered that there was heterogeneity in the degree of tumor growth inhibition (TGI) observed when tumors were tested against single agent gefitinib. In 7% ( 6/86) of tumors considerable inhibition of tumor growth was observed, but most showed a more modest response resulting in a low degree of TGI. Interestingly, gefitinib had both positive and negative effects when used in combination with different cytotoxic drugs. In 59% ( 45/76) of tumors tested, the addition of gefitinib appeared to potentiate the effect of the cytotoxic agent or combination (of these, 11% ( 5/45) had a >50% decrease in their IndexSUM). In 38% of tumors ( 29/76), the TGI was decreased when the combination of gefitinib+cytotoxic drug was used in comparison to the cytotoxic drug alone. In the remaining 3% ( 2/76) there was no change observed.

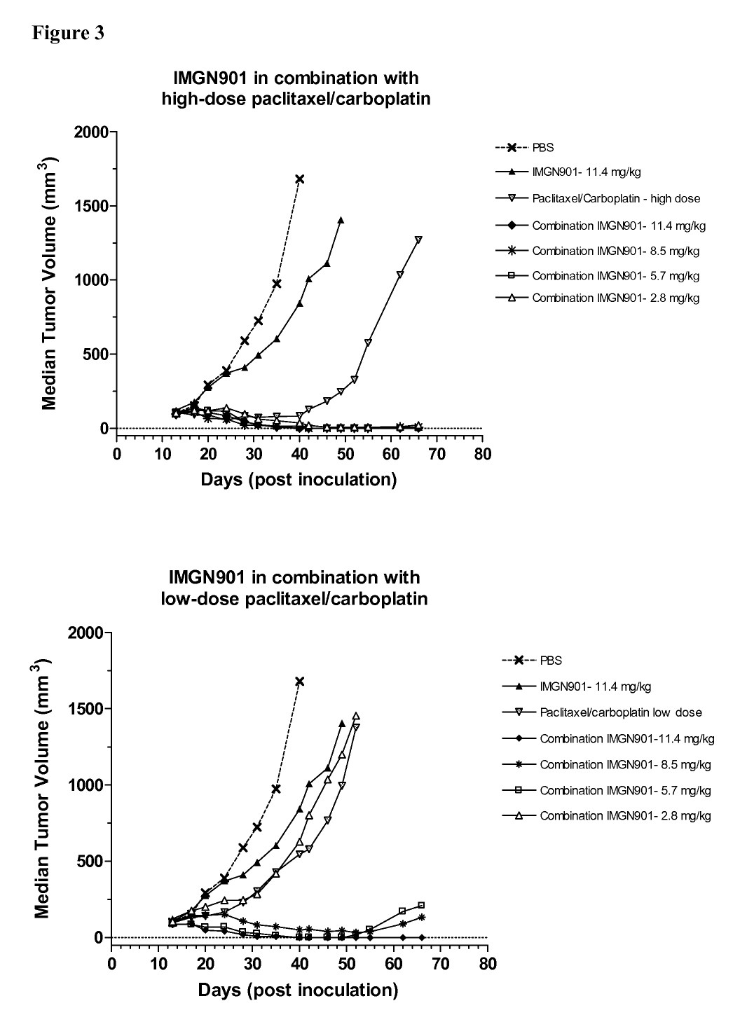

The authors conclude that gefitinib in combination with different cytotoxic agents (cisplatin; gemcitabine; oxaliplatin; treosulfan and treosulfan+gemcitabine) is a double-edged sword: their effect on growth rate may make some tumors more resistant to concomitant cytotoxic chemotherapy, while their effect on cytokine-mediated cell survival (anti-apoptotic) mechanisms may potentiate sensitivity to the same drugs in tumors from other individuals. See, conclusion on page 7; see also FIG. 3. Knight et al., BMC Cancer 2004, 4:83. Thus, this study proves that when two compounds, which are known to be useful for the same purpose, are combined for that purpose, they may not necessarily perform as expected.

Finding highly efficacious combinations, i.e., synergistic mixtures, of active agents is a challenging endeavor. Serendipity is not a valid route as the number of potential combinations of agents is staggeringly large. For example, there are trillions of possible 5 fold combinations of even a relatively small palette of 5000 potential agents. The other normal discovery strategy of deducing potential combinations from knowledge of mechanism is also limited in its potential because many biological end points of living organisms are affected by multiple pathways. These pathways are often not known, and even when they are, the ways in which the pathways interact to produce the biological end effect are often unknown.

Synergistic uses of combinations of drugs even if previously demonstrated do not obviate the need to look for new synergistic combinations because synergistic effects are unpredictable. For example, in treatment of autoimmune deficiency syndrome (AIDS), which involved highly active anti-retroviral therapy (HAART), it was believed that cocktail of inhibitors of HIV-1 viral reverse transcriptase (RT) and the viral protease (PR), exhibit synergistic inhibition of viral replication. Later on, intriguingly, synergy was also observed within two classes of RT inhibitors—that is, the nucleoside RT inhibitors (NRTIs) showed synergy with the nonnucleoside RT inhibitors (NNRTIs) in the absence of PR inhibitors. For example, NRTI, AZT (zidovudine) and the NNRTI, nevirapin exhibit synergy when given in combination (Basavapathruni A et al., J. Biol. Chem., Vol. 279, Issue 8, 6221-6224, Feb. 20, 2004). Thus, there is still a need for finding drug combinations that show synergism and can be effectively used for the treatment and prevention of debilitating diseases, particularly with respect to treatment of particular types of cancer, such as ovarian cancer.

In one embodiment of the invention, it has surprisingly been discovered that a pharmaceutical composition comprising a combination of a CD56-binding immunoconjugate, a taxane compound, and a platinum compound produce a synergistic therapeutic effect in the treatment of ovarian cancer.

The term “synergistic effect”, as used herein, refers to a greater-than-additive therapeutic effect produced by a combination of compounds wherein the therapeutic effect obtained with the combination exceeds the additive effects that would otherwise result from individual administration the compounds alone. Embodiments of the invention include methods of producing a synergistic effect in the treatment of ovarian cancer, wherein said effect is at least 5%, at least 10%, at least 20%, at least 30%, at least 40%, at least 50%, at least 60%, at least 70%, at least 80%, at least 90%, at least 100%, at least 200%, at least 500%, or at least 1000% greater than the corresponding additive effect.

In some embodiments, a synergistic effect is obtained in the treatment of ovarian cancer wherein one or more of the agents or compounds are administered in a “low dose” (i.e., using a dose or doses which would be considered non-therapeutic if administered alone), wherein the administration of the low dose compound or agent in combination with other compounds or agents (administered at either a low or therapeutic dose) results in a synergistic effect which exceeds the additive effects that would otherwise result from individual administration the compounds alone. In some embodiments, the synergistic effect is achieved via administration of one or more of the agents or compounds administered in a “low dose” wherein the low dose is provided to reduce or avoid toxicity or other undesirable side effects.

In one embodiment, a synergistic effect is obtained in the treatment of ovarian cancer wherein one or more of the agents or compounds administered in a low dose comprise any one of, or any combination of one or more of, IMGN901, paclitaxel, and/or carboplatin. In another embodiment, a synergistic effect is obtained in the treatment of ovarian cancer wherein the agents or compounds administered comprise low dose IMGN901, low dose paclitaxel, and low dose carboplatin.

### CD56 Antibodies and Fragments Thereof

Antibodies that specifically bind CD56 (i.e., “CD56 antibodies”) used in the present invention include any type of CD56 antibody or CD56-binding fragments, portions, or other antigen binding forms thereof. These include, for example, but without limitation various forms of antibodies and fragments thereof such as:
Antibodies and derivatives or analogues thereof such as—
   polyclonal or monoclonal antibodies or antigen-binding fragments thereof;
   chimeric, primatized, humanized, fully human antibodies or antigen-binding fragments thereof;
   resurfaced antibodies or antigen-binding fragments thereof (see, e.g., U.S. Pat. No. 5,639,641);
   epitope binding fragments of antibodies such as single-chain, Fv, sFv, scFv, Fab, Fab′, and F(ab′)2 (Parham, J. Immunol. 131:2895-2902 (1983); Spring et al, J. Immunol. 113:470-478 (1974); Nisonoff et al, Arch. Biochem. Biophys. 89:230-244 (1960)).

Additional examples of the broad variety and nature of types of antigen binding molecules that may be generated and used as CD56-binding agents are discussed in further detail subsequently herein.

### IMGN901

The antibody portion of IMGN901 was originally derived from N901. N901 is an IgG1 murine monoclonal antibody (also called anti-N901) that is reactive with CD56, which is expressed on tumors of neuroendocrine origin. See e.g., Griffin et al, J. Immunol. 130:2947-2951 (1983) and U.S. Pat. No. 5,639,641.

The CD56 antigen is a neural cell adhesion molecule (NCAM) that is expressed on the surface of tumor cells of neuroendocrine origin, including small cell lung carcinomas (SCLC), carcinoid tumors and Merkel cell carcinomas (MCC). CD56 is expressed on approximately 56% of ovarian tumors. CD56 is also expressed on approximately 70% of multiple myelomas.

The preparation of different versions of humanized N901, is described, for example, by Roguska et al, Proc. Natl. Acad. Sci. USA, 91:969-973 (1994), and Roguska et al, Protein Eng., 9:895:904 (1996), the disclosures of which are incorporated by reference herein in their entirety. To denote a humanized antibody, the letters “hu” or “h” appear before the name of the antibody. For example, humanized N901 may be referred to as huN901 or hN901.

IMGN901 is an antibody-drug conjugate (ADC) comprised of the CD56-binding monoclonal antibody, huN901, and the maytansinoid cytotoxic agent, DM1. See, U.S. Pat. No. 7,303,749, Example 1, for an exemplary description of huN901/DM1 conjugation. The entirety of U.S. Pat. No. 7,303,749 (Inventor: R. V. J. Chari; Issued Dec. 4, 2007) is incorporated by reference herein. Additional information regarding maytansinoid compounds is also discussed further herein.

IMGN901 binds with high affinity to CD56 expressed on the surface of tumor cells. Once bound, the conjugate is internalized and the DM1 is released.

DM1 is an antimitotic agent that disrupts tubulin polymerization and microtubule assembly. See, Remillard S. et al., 1975, Science 189:1002-1005). See also, U.S. Pat. No. 7,303,749, Example 1, describing that “Ansamitocin P-3, provided by Takeda (Osaka, Japan) was converted to the disulfide-containing maytansinoid DM1, as described herein and in U.S. Pat. No. 5,208,020.” The entirety of U.S. Pat. No. 5,208,020 (Inventors: Chari et al.; Issued May 4, 1993) is incorporated by reference herein.

IMGN901 shows marked antitumor activity as a single agent in human xenograft preclinical models for ovarian cancer.

### Maytansinoids and Other Anti-Mitotic Agents

A mitotic inhibitor (anti-mitotic agent) is a type of drug commonly derived from natural substances such as plant alkaloids which are often used in cancer treatment and cytogenetic research. Cancer cells grow, and eventually metastasize, through continuous mitotic division. Generally, mitotic inhibitors prevent cells from undergoing mitosis by disrupting microtubule polymerization, thus preventing cancerous growth. Mitotic inhibitors work by interfering with and halting mitosis (usually during the M phase of the cell cycle), so that a cell can no longer divide. Polymerization of tubulin, which is necessary for mitosis to occur, may be suppressed by mitotic inhibitors, thereby preventing mitosis. Some examples of mitotic inhibitors used in the treatment of cancer include the maytansanoid DM1, paclitaxel, docetaxel, vinblastine, vincristine, and vinorelbine.

Maytansinoids that can be used in the present invention are well known in the art and can be isolated from natural sources according to known methods or prepared synthetically according to known methods. Examples of suitable maytansinoids include maytansinol and maytansinol analogues. Examples of suitable maytansinol analogues include those having a modified aromatic ring and those having modifications at other positions.

Some specific examples of suitable analogues of maytansinol having a modified aromatic ring include:
(1) C-19-dechloro (U.S. Pat. No. 4,256,746) (prepared by LAH reduction of ansamitocin P2);
(2) C-20-hydroxy (or C-20-demethyl) +/−C-19-dechloro (U.S. Pat. Nos. 4,361,650 and 4,307,016) (prepared by demethylation using Streptomyces or Actinomyces or dechlorination using LAH); and
(3) C-20-demethoxy, C-20-acyloxy (—OCOR), +/−dechloro (U.S. Pat. No. 4,294,757) (prepared by acylation using acyl chlorides).

Some specific examples of suitable analogues of maytansinol having modifications of other positions include:
(1) C-9-SH (U.S. Pat. No. 4,424,219) (prepared by the reaction of maytansinol with H₂S or P₂S₅);
(2) C-14-alkoxymethyl (demethoxy/CH₂OR) (U.S. Pat. No. 4,331,598);
(3) C-14-hydroxymethyl or acyloxymethyl (CH₂OH or CH₂OAc) (U.S. Pat. No. 4,450,254) (prepared from Nocardia);
(4) C-15-hydroxy/acyloxy (U.S. Pat. No. 4,364,866) (prepared by the conversion of maytansinol by Streptomyces);
(5) C-15-methoxy (U.S. Pat. Nos. 4,313,946 and 4,315,929) (isolated from Trewia nudiflora);
(6) C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

A synthesis of thiol-containing maytansinoids useful in the present invention is disclosed in U.S. Pat. Nos. 5,208,020; 5,416,064; 6,333,410; 7,276,497; and 7,301,019.

Maytansinoids with a thiol moiety at the C-3 position, the C-14 position, the C-15 position or the C-20 position are all expected to be useful. The C-3 position is preferred and the C-3 position of maytansinol is especially preferred. Also preferred are an N-methyl-alanine-containing C-3 thiol moiety maytansinoid, and an N-methyl-cysteine-containing C-3 thiol moiety maytansinoid, and analogues of each.

Some specific examples of N-methyl-alanine-containing C-3 thiol moiety maytansinoid derivatives useful in the present invention are represented by the formula M1, M2, M3, M6 and M7.

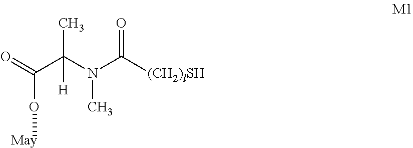

wherein:
l is an integer of from 1 to 10; and
may is a maytansinoid.

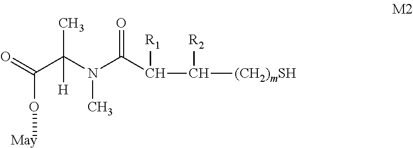

wherein:
R₁ and R₂ are H, CH₃ or CH₂CH₃, and may be the same or different;
m is 0, 1, 2 or 3; and
may is a maytansinoid.

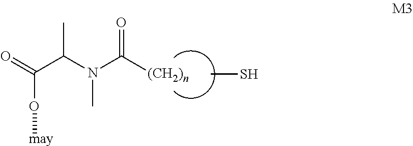

wherein:
n is an integer of from 3 to 8; and
may is a maytansinoid.

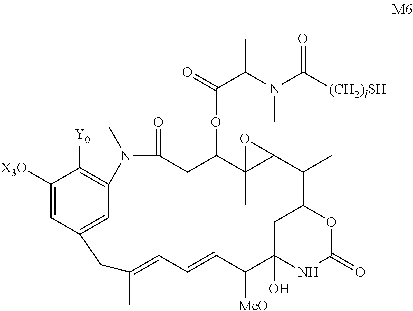

wherein:
l is 1, 2 or 3;

### Y₀ is Cl or H; and

### X₃ is H or CH₃.

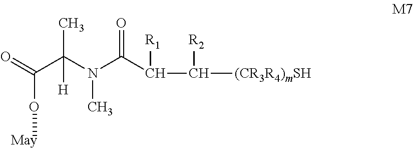

wherein:
R₁, R₂, R₃, R₄ are H, CH₃ or CH₂CH₃, and may be the same or different;
m is 0, 1, 2 or 3; and
may is a maytansinoid.

Some specific examples of N-methyl-cysteine-containing C-3 thiol moiety maytansinoid derivatives useful in the present invention are represented by the formula M4 and M5.

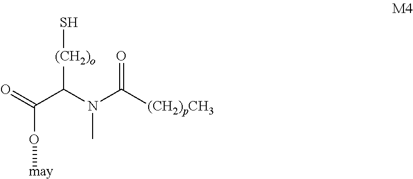

wherein:
o is 1, 2 or 3;
p is an integer of 0 to 10; and
may is a maytansinoid.

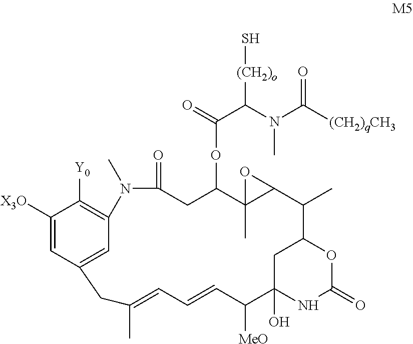

wherein:

o is 1, 2 or 3;

q is an integer of from 0 to 10;

Y₀ is Cl or H; and

X₃ is H or CH₃.

Some embodiments of maytansinoids are also described in U.S. Pat. Nos. 5,208,020; 5,416,064; 6,333,410; 6,441,163; 6,716,821; RE39,151; and 7,276,497.

In one embodiment of the invention a pharmaceutical composition used in the treatment of ovarian cancer comprises IMGN901, one or both of paclitaxel and docetaxel, and one or any combination of carboplatin, cisplatin, and oxaliplatin. In one embodiment of the invention a pharmaceutical composition used in the treatment of ovarian cancer comprises IMGN901, paclitaxel and carboplatin.

### Conjugate Linkage

A cell-binding agent of the invention may be modified by reacting a bifunctional crosslinking reagent with the cell-binding agent, thereby resulting in the covalent attachment of a linker molecule to the cell-binding agent. As used herein, a “bifunctional crosslinking reagent” is any chemical moiety that covalently links a cell-binding agent to a drug, such as the drugs described herein. In a preferred embodiment of the invention, a portion of the linking moiety is provided by the drug. In this respect, the drug comprises a linking moiety that is part of a larger linker molecule that is used to join the cell-binding agent to the drug. For example, to form the maytansinoid DM1 or DM4, the ester side chain at the C-3 position of maytansine is modified to have a free sulfhydryl group (SH), as described in U.S. Pat. Nos. 5,208,020; 6,333,410; and 7,276,497. This thiolated form of maytansine can react with a modified cell-binding agent to form a conjugate. Therefore, the final linker is assembled from two components, one of which is provided by the crosslinking reagent, while the other is provided by the side chain from DM1 or DM4.

Any suitable bifunctional crosslinking reagent can be used in connection with the invention, so long as the linker reagent provides for retention of the therapeutic (e.g., cytotoxicity), and targeting characteristics of the drug and the cell-binding agent, respectively. Preferably, the linker molecule joins the drug to the cell-binding agent through chemical bonds (as described above), such that the drug and the cell-binding agent are chemically coupled (e.g., covalently bonded) to each other. Preferably, the linking reagent is a cleavable linker. More preferably, the linker is cleaved under mild conditions, i.e., conditions within a cell under which the activity of the drug is not affected. Examples of suitable cleavable linkers include disulfide linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)).

In one embodiment, a cytotoxic compound is linked to a cell-binding agent through a disulfide bond or a thioether bond. The linker molecule comprises a reactive chemical group that can react with the cell-binding agent. Exemplary reactive chemical groups for reaction with the cell-binding agent are N-succinimidyl esters and N-sulfosuccinimidyl esters. Additionally the linker molecule may comprise a reactive chemical group, such as a dithiopyridyl group that can react with the drug to form a disulfide bond. Particular embodiments of linker molecules include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), and other reactive cross-linkers which are described in U.S. Pat. No. 6,913,748.

Embodiments of the invention include both cleavable linkers and non-cleavable linker to generate the above-described conjugate. A non-cleavable linker is any chemical moiety that is capable of linking a drug, such as a maytansinoid, a Vinca alkaloid, a dolastatin, an auristatin, or a cryptophycin, to a cell-binding agent in a stable, covalent manner. Non-cleavable linkers are substantially resistant to acid-induced cleavage, light-induced cleavage, peptidase-induced cleavage, esterase-induced cleavage, and disulfide bond cleavage, at conditions under which the drug or the cell-binding agent remains active.

Suitable crosslinking reagents that form non-cleavable linkers between a drug and the cell-binding agent are well known in the art. Examples of non-cleavable linkers include linkers having an N-succinimidyl ester or N-sulfosuccinimidyl ester moiety for reaction with the cell-binding agent, as well as a maleimido- or haloacetyl-based moiety for reaction with the drug. Crosslinking reagents comprising a maleimido-based moiety include N-succinimidyl 4-(maleimidomethyl)cyclohexanecarboxylate (SMCC), N-succinimidyl-4-(N-maleimidomethyl)-cyclohexane-1-carboxy-(6-amido caproate), which is a “long chain” analog of SMCC (LC-SMCC), kappa-maleimidoundecanoic acid N-succinimidyl ester (KMUA), gamma-maleimidobutyric acid N-succinimidyl ester (GMBS), epsilon-maleimidocaproic acid N-hydroxysuccinimide ester (EMCS), m-maleimidobenzoyl-N-hydroxysuccinimide ester (MBS), N-(alpha-maleimidoacetoxy)-succinimide ester (AMAS), succinimidyl-6-(beta-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

Other crosslinking reagents lacking a sulfur atom that form non-cleavable linkers may also be used as embodiments of the invention. Such linkers can be derived, for example, from dicarboxylic acid based moieties. Suitable dicarboxylic acid based moieties include, but are not limited to, α,ω-dicarboxylic acids of the general formula (IX):

HOOC—X₁—Yₙ—Zₘ—COOH (IX),

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Exemplary non-cleavable linkers disclosed herein are described in U.S. patent application Ser. No. 10/960,602 (U.S. Publication No. 2005/0169933). Other linkers which can be used in the present invention include charged linkers or hydrophilic linkers and are described in U.S. patent application Nos. 12/433,604 (U.S. Publication No. 2009/0274713) and 12/574,466 (U.S. Publication No. 2010/0129314), respectively.

Alternatively, as disclosed in U.S. Pat. No. 6,441,163 B1, the drug can be first modified to introduce a reactive ester suitable to react with a cell-binding agent. Reaction of these maytansinoids containing an activated linker moiety with a cell-binding agent provides another method of producing a cleavable or non-cleavable cell-binding agent maytansinoid conjugate.

### Taxanes

Taxane compounds prevent the growth of cancer cells by affecting cell structures called microtubules, which play critical roles in cell functions. During normal cell growth, microtubules are formed when a cell starts dividing. Once a cell stops dividing, the microtubules are broken down or destroyed. Taxane compounds stop the microtubules from breaking down, such that the cancer cells become clogged with microtubules such that they cannot continue to grow and divide.

Taxane compounds are well-known in the art and include, for example, paclitaxel (available as TAXOL® from Bristol-Myers Squibb, Princeton, N.J.) and docetaxel (available as TAXOTERE® from Sanofi-Aventis (U.S), Bridgewater, N.J.), etc. Other taxane compounds that become approved by the U.S. Food and Drug Administration (FDA) or foreign counterparts thereof are also contemplated for use in the methods and compositions of the present invention. Other taxane compounds that can be used in the present invention include those described, for example, in 10th NCI-EORTC Symposium on New Drugs in Cancer Therapy, Amsterdam, page 100, Nos. 382 and 383 (Jun. 16-19, 1998); and U.S. Pat. Nos. 4,814,470, 5,721,268, 5,714,513, 5,739,362, 5,728,850, 5,728,725, 5,710,287, 5,637,484, 5,629,433, 5,580,899, 5,549,830, 5,523,219, 5,281,727, 5,939,567, 5,703,117, 5,480,639, 5,250,683, 5,700,669, 5,665,576, 5,618,538, 5,279,953, 5,243,045, 5,654,447, 5,527,702, 5,415,869, 5,279,949, 5,739,016, 5,698,582, 5,478,736, 5,227,400, 5,516,676, 5,489,601, 5,908,759, 5,760,251, 5,578,739, 5,547,981, 5,547,866, 5,344,775, 5,338,872, 5,717,115, 5,620,875, 5,284,865, 5,284,864, 5,254,703, 5,202,448, 5,723,634, 5,654,448, 5,466,834, 5,430,160, 5,407,816, 5,283,253, 5,719,177, 5,670,663, 5,616,330, 5,561,055, 5,449,790, 5,405,972, 5,380,916, 5,912,263, 8,808,113, 5,703,247, 5,618,952, 5,367,086, 5,200,534, 5,763,628, 5,705,508, 5,622,986, 5,476,954, 5,475,120, 5,412,116, 5,916,783, 5,879,929, 5,861,515, 5,795,909, 5,760,252, 5,637,732, 5,614,645, 5,599,820, 5,310,672, RE 34,277, 5,877,205, 5,808,102, 5,766,635, 5,760,219, 5,750,561, 5,637,723, 5,475,011, 5,256,801, 5,900,367, 5,869,680, 5,728,687, 5,565,478, 5,411,984, 5,334,732, 5,919,815, 5,912,264, 5,773,464, 5,670,673, 5,635,531, 5,508,447, 5,919,816, 5,908,835, 5,902,822, 5,880,131, 5,861,302, 5,850,032, 5,824,701, 5,817,867, 5,811,292, 5,763,477, 5,756,776, 5,686,623, 5,646,176, 5,621,121, 5,616,739, 5,602,272, 5,587,489, 5,567,614, 5,498,738, 5,438,072, 5,403,858, 5,356,928, 5,274,137, 5,019,504, 5,917,062, 5,892,063, 5,840,930, 5,840,900, 5,821,263, 5,756,301, 5,750,738, 5,750,562, 5,726,318, 5,714,512, 5,686,298, 5,684,168, 5,681,970, 5,679,807, 5,648,505, 5,641,803, 5,606,083, 5,599,942, 5,420,337, 5,407,674, 5,399,726, 5,322,779, 4,924,011, 5,939,566, 5,939,561, 5,935,955, 5,919,455, 5,854,278, 5,854,178, 5,840,929, 5,840,748, 5,821,363, 5,817,321, 5,814,658, 5,807,888, 5,792,877, 5,780,653, 5,770,745, 5,767,282, 5,739,359, 5,726,346, 5,717,103, 5,710,099, 5,698,712, 5,683,715, 5,677,462, 5,670,653, 5,665,761, 5,654,328, 5,643,575, 5,621,001, 5,608,102, 5,606,068, 5,587,493, 5,580,998, 5,580,997, 5,576,450, 5,574,156, 5,571,917, 5,556,878, 5,550,261, 5,539,103, 5,532,388, 5,470,866, 5,453,520, 5,384,399, 5,364,947, 5,350,866, 5,336,684, 5,296,506, 5,290,957, 5,274,124, 5,264,591, 5,250,722, 5,229,526, 5,175,315, 5,136,060, 5,015,744, 4,924,012, 6,118,011, 6,114,365, 6,107,332, 6,072,060, 6,066,749, 6,066,747, 6,051,724, 6,051,600, 6,048,990, 6,040,330, 6,030,818, 6,028,205, 6,025,516, 6,025,385, 6,018,073, 6,017,935, 6,011,056, 6,005,138, 6,005,138, 6,005,120, 6,002,023, 5,998,656, 5,994,576, 5,981,564, 5,977,386, 5,977,163, 5,965,739, 5,955,489, 5,939,567, 5,939,566, 5,919,815, 5,912,264, 5,912,263, 5,908,835, and 5,902,822.

Other compounds that can be used in the invention are those that act through a taxane-like mechanism. Compounds that act through a taxane-like mechanism include compounds that have the ability to exert microtubule-stabilizing effects and cytotoxic activity against rapidly proliferating cells, such as tumor cells or other hyperproliferative cellular diseases. Such compounds include, for example, epothilone compounds, such as, for example, epothilone A, B, C, D, E and F, and derivatives thereof. Other compounds that act through a taxane-like mechanism (e.g., epothilone compounds) that become approved by the FDA or foreign counterparts thereof are also preferred for use in the methods and compositions of the present invention. Epothilone compounds and derivatives thereof are known in the art and are described, for example, in U.S. Pat. Nos. 6,121,029; 6,117,659; 6,096,757; 6,043,372; 5,969,145; 5,886,026; and in PCT Application Nos.: WO 97/19086; WO 98/08849; WO 98/22461; WO 98/25929; WO 98/38192; WO 99/01124; WO 99/02514; WO 99/03848; WO 99/07692; WO 99/27890; and WO 99/28324.

Platinum Compounds

Platinum compounds that may be used as one component in embodiments of the invention include, for example, cisplatin (available as PLATINOL® from Bristol-Myers Squibb, Princeton, N.J.), carboplatin (available as PARAPLATIN® from Bristol-Myers Squibb, Princeton, N.J.), oxaliplatin (available as ELOXATINE® from Sanofi-Aventis (U.S), Bridgewater, N.J.), iproplatin, ormaplatin, and tetraplatin, etc. Other platinum compounds that become approved by the FDA or foreign counterparts thereof are also contemplated for use in the methods and compositions of the present invention. Platinum compounds that are useful in treating cancer are known in the art and are described, for example in U.S. Pat. Nos. 4,994,591, 4,906,646, 5,902,610, 5,053,226, 5,789,000, 5,871,710, 5,561,042, 5,604,095, 5,849,790, 5,705,334, 4,863,902, 4,767,611, 5,670,621, 5,384,127, 5,084,002, 4,937,262, 5,882,941, 5,879,917, 5,434,256, 5,393,909, 5,117,022, 5,041,578, 5,843,475, 5,633,243, 5,178,876, 5,866,169, 5,846,725, 5,646,011, 5,527,905, 5,844,001, 5,832,931, 5,676,978, 5,604,112, 5,562,925, 5,541,232, 5,426,203, 5,288,887, 5,041,581, 5,002,755, 4,946,954, 4,921,963, 4,895,936, 4,686,104, 4,594,238, 4,581,224, 4,250,189, 5,829,448, 5,690,905, 5,665,771, 5,648,384, 5,633,016, 5,460,785, 5,395,947, 5,256,653, 5,132,323, 5,130,308, 5,106,974, 5,059,591, 5,026,694, 4,992,553, 4,956,459, 4,956,454, 4,952,676, 4,895,935, 4,892,735, 4,843,161, 4,760,156, 4,739,087, 4,720,504, 4,544,759, 4,515,954, 4,466,924, 4,462,998, 4,457,926, 4,428,943, 4,325,950, 4,291,027, 4,291,023, 4,284,579, 4,271,085, 4,234,500, 4,234,499, 4,200,583, 4,175,133, 4,169,846, 5,922,741, 5,922,674, 5,922,302, 5,919,126, 5,910,102, 5,876,693, 5,871,923, 5,866,617, 5,866,615, 5,866,593, 5,864,024, 5,861,139, 5,859,034, 5,855,867, 5,855,748, 5,849,770, 5,843,993, 5,824,664, 5,821,453, 5,811,119, 5,798,373, 5,786,354, 5,780,478, 5,780,477, 5,776,925, 5,770,593, 5,770,222, 5,747,534, 5,739,144, 5,738,838, 5,736,156, 5,736,119, 5,723,460, 5,697,902, 5,693,659, 5,688,773, 5,674,880, 5,670,627, 5,665,343, 5,654,287, 5,648,362, 5,646,124, 5,641,627, 5,635,218, 5,633,257, 5,632,982, 5,622,977, 5,622,686, 5,618,393, 5,616,613, 5,612,019, 5,608,070, 5,595,878, 5,585,112, 5,580,888, 5,580,575, 5,578,590, 5,575,749, 5,573,761, 5,571,153, 5,563,132, 5,561,136, 5,556,609, 5,552,156, 5,547,982, 5,542,935, 5,525,338, 5,519,155, 5,498,227, 5,491,147, 5,482,698, 5,469,854, 5,455,270, 5,443,816, 5,415,869, 5,409,915, 5,409,893, 5,409,677, 5,399,694, 5,399,363, 5,380,897, 5,340,565, 5,324,591, 5,318,962, 5,302,587, 5,292,497, 5,272,056, 5,258,376, 5,238,955, 5,237,064, 5,213,788, 5,204,107, 5,194,645, 5,182,368, 5,130,145, 5,116,831, 5,106,858, 5,100,877, 5,087,712, 5,087,618, 5,078,137, 5,057,302, 5,049,396, 5,034,552, 5,028,726, 5,011,846, 5,010,103, 4,985,416, 4,970,324, 4,936,465, 4,931,553, 4,927,966, 4,912,072, 4,906,755, 4,897,384, 4,880,832, 4,871,528, 4,822,892, 4,783,452, 4,767,874, 4,760,155, 4,687,780, 4,671,958, 4,665,210, 4,645,661, 4,599,352, 4,594,418, 4,593,034, 4,587,331, 4,575,550, 4,562,275, 4,550,169, 4,482,569, 4,431,666, 4,419,351, 4,407,300, 4,394,319, 4,335,087, 4,329,299, 4,322,391, 4,302,446, 4,287,187, 4,278,660, 4,273,755, 4,255,417, 4,255,347, 4,248,840, 4,225,529, 4,207,416, 4,203,912, 4,177,263, 4,151,185, 4,140,707, 4,137,248, 4,115,418, 4,079,121, 4,075,307, 3,983,118, 3,870,719, RE 33,071, 6,087,392, 6,077,864, 5,998,648, and 5,902,610.

### Dosing and Administration

Embodiments of the invention include immunoconjugates and cytotoxic compounds/chemotherapeutic agents used with pharmaceutically acceptable carriers, diluents, and/or excipients, which are well known, and can be determined, by one of skill in the art as the clinical situation warrants. Examples of suitable carriers, diluents and/or excipients include: (1) Dulbecco's phosphate buffered saline, pH about 6.5, which would contain about 1 mg/ml to 25 mg/ml human serum albumin, (2) 0.9% saline (0.9% w/v NaCl), and (3) 5% (w/v) dextrose.

Compounds and compositions described herein may be administered in appropriate forms and via routes such as would be used by one of skill in the art. Some examples of various possible modes of administration include, without limitation, parenteral, intravenous, intraarterial, intraperitoneal, subcutaneous, intramuscular, intradermal. For various modes of administration, the compounds or compositions can be aqueous or nonaqueous sterile solutions, suspensions or emulsions. Propylene glycol, vegetable oils and injectable organic esters, such as ethyl oleate, can be used as the solvent or vehicle. The compositions can also contain adjuvants, emulsifiers or dispersants. Compositions can also be in the form of sterile solid compositions which can be dissolved or dispersed in sterile water or any other injectable sterile medium.

Pharmaceutical compositions may be administered in any order or at any interval as determined by one of skill in the art. For example, but without limitation, a CD56-binding agent linked to a cytotoxic compound (such as IMGN901), a taxane compound (such as paclitaxel), and a platinum compound (such as carboplatin) may be administered sequentially (in any order), simultaneously, or via any combination of sequential and simultaneous administrations (such as, in one of many possible examples, by simultaneous administration of taxane and platinum compounds, followed at a desired interval thereafter by administration of a CD56-binding agent linked to a cytotoxic compound). Any combination of sequential or simultaneous administration protocols may be used and implemented as decided and determined by one of skill in the art. Administration of pharmaceutical compounds, whether simultaneous, sequential or a combination of both, may be performed according to any number of desired intervals of minutes (e.g., 0-60 minutes), hours (e.g., 0-24 hours), days (e.g., 0-7 days), and/or weeks (e.g., 0-52 weeks) as may be decided and determined by one of skill in the art.

A “therapeutically effective amount” of the chemotherapeutic agents and immunoconjugates described herein refers to the dosage regimen for inhibiting the proliferation of selected cell populations and/or treating a patient's disease, and is selected in accordance with a variety of factors, including the age, weight, sex, diet and medical condition of the patient, the severity of the disease, the route of administration, and pharmacological considerations, such as the activity, efficacy, pharmacokinetic and toxicology profiles of the particular compound used. The “therapeutically effective amount” can also be determined by reference to standard medical texts, such as the Physicians Desk Reference 2010 (Publisher: PDR Network, LLC; ISBN-10: 1563637480; ISBN-13: 978-1563637483). Embodiments of the invention include methods of treating ovarian cancer in human and non-human mammals.

Examples of suitable protocols of administration of pharmaceutical/therapeutic compositions of the invention may be considered, without limitation, to include parameters such as follows. Pharmaceutical compositions may be given daily for about 5 days either as an i.v. bolus each day for about 5 days, or as a continuous infusion for about 5 days.

Pharmaceutical compositions may be administered once a week for six weeks or longer. Pharmaceutical compositions may be administered once every two or three weeks. Bolus doses may be given in about 50 to about 400 ml of normal saline to which about 5 to about 10 ml of human serum albumin can be added. Continuous infusions may be given in about 250 to about 500 ml of normal saline, to which about 25 to about 50 ml of human serum albumin can be added, per 24 hour period. Dosages may be about 10 pg to about 1000 mg/kg per person, i.v. (range of about 100 ng to about 10 mg/kg).

About one to about four weeks after treatment, a patient may receive a second course of treatment. Specific clinical protocols with regard to route of administration, excipients, diluents, dosages, and times can be determined by the skilled artisan as the clinical situation warrants.

The present invention also provides pharmaceutical kits comprising one or more containers filled with one or more of the ingredients of the pharmaceutical compounds and/or compositions of the present invention, including, one or more immunoconjugates and one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products.

Cancer therapies and their dosages, routes of administration and recommended usage are known in the art and have been described in such literature as the Physician's Desk Reference (PDR). The PDR discloses dosages of the agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician. The contents of the PDR are expressly incorporated herein in its entirety by reference. The 2006 edition of the Physician's Desk Reference (PDR) discloses the mechanism of action and preferred doses of treatment and dosing schedules for thalidomide (p 979-983) Velcade (p 2102-2106) and melphalan (p 976-979). The contents of the PDR are expressly incorporated herein in their entirety by reference. One of skill in the art can review the PDR, using one or more of the following parameters, to determine dosing regimen and dosages of the chemotherapeutic agents and conjugates that can be used in accordance with the teachings of this invention. These parameters include:
1. Comprehensive index
   a) by Manufacturer
   b) Products (by company's or trademarked drug name)
   c) Category index (for example, “proteasome inhibitors”, “DNA alkylating agents,” “melphalan” etc.)
   d) Generic/chemical index (non-trademark common drug names)
2. Color images of medications
3. Product information, consistent with FDA labeling
   a) Chemical information
   b) Function/action
   c) Indications & Contraindications
   d) Trial research, side effects, warnings

### Analogues and Derivatives

One skilled in the art of therapeutic agents, such as cytotoxic agents or chemotherapeutic agents, will readily understand that each of the such agents described herein can be modified in such a manner that the resulting compound still retains the specificity and/or activity of the starting compound. The skilled artisan will also understand that many of these compounds can be used in place of the therapeutic agents described herein. Thus, the therapeutic agents of the present invention include analogues and derivatives of the compounds described herein.

### Immunoglobulins and Antibodies

The terms “antibody” and “immunoglobulin” may be used interchangeably herein. An antibody or immunoglobulin comprises at least the variable domain of a heavy chain, and normally comprises at least the variable domains of a heavy chain and a light chain. Basic immunoglobulin structures in vertebrate systems are well understood to those of ordinary skill in the art. See, e.g., Harlow et al., Antibodies: A Laboratory Manual, (Cold Spring Harbor Laboratory Press, 2nd ed. 1988).

The term “immunoglobulin” comprises various broad classes of polypeptides that can be distinguished biochemically. Those skilled in the art will appreciate that heavy chains are classified as gamma, mu, alpha, delta, or epsilon, with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention. As one example, a typical IgG immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The N-terminal portion is a variable region and at the C-terminal portion is a constant region; the CH3 and CL domains actually comprise the carboxy-terminus of the heavy and light chain, respectively.

Variable regions allow the antibodies to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers Casterman et al., Nature 363:446 448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to CD56 antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

The term “specifically binds,” generally means that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “1” may be deemed to have a higher specificity for a given epitope than antibody “2,” or antibody “1” may be said to bind to epitope “3” with a higher specificity than it has for related epitope “4.”

Monoclonal antibodies may be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. For example, monoclonal antibodies can be prepared using CD56 knockout mice to increase the regions of epitope recognition. Monoclonal antibodies can be prepared using a wide variety of techniques known in the art including the use of hybridoma and recombinant and phage display technology as described elsewhere herein.

Antibody fragments that recognize specific epitopes may be generated by known techniques. For example, Fab and F(ab′)2 fragments may be produced recombinantly or by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

Those skilled in the art will also appreciate that DNA encoding antibodies or antibody fragments (e.g., antigen binding sites) may also be derived from antibody libraries, such as phage display libraries. In particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules from a non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(⅘):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art, such as for example but without limitation including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741. Human antibodies can also be produced using transgenic mice which are incapable of expressing functional endogenous immunoglobulins, but which can express human immunoglobulin genes. See e.g., Lonberg and Huszar, Int. Rev. Immunol. 13:65-93 (1995); WO 98/24893; WO 96/34096; WO 96/33735; U.S. Pat. Nos. 5,413,923; 5,625,126; 5,633,425; 5,569,825; 5,661,016; 5,545,806; and 5,814,318.

Monoclonal antibody techniques allow for the production of specific cell-binding agents in the form of monoclonal antibodies. Particularly well known in the art are techniques for creating monoclonal antibodies produced by immunizing mice, rats, hamsters or any other mammal with the antigen of interest such as the intact target cell, antigens isolated from the target cell, whole virus, attenuated whole virus, and viral proteins such as viral coat proteins. Sensitized human cells can also be used. Another method of creating monoclonal antibodies is the use of phage libraries of sFv (single chain variable region), specifically human sFv (see, e.g., Griffiths et al, U.S. Pat. No. 5,885,793; McCafferty et al, WO 92/01047; Liming et al, WO 99/06587.)

Selection of the appropriate cell-binding agent is a matter of choice that depends upon the particular cell population that is to be targeted, but in general monoclonal antibodies and epitope binding fragments thereof are preferred, if an appropriate one is available.

### Additional Guides to Methods and Techniques

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical Immunology (8th ed.), Appleton & Lange, Norwalk, Conn. (1994) and Mishell and Shiigi (eds), Selected Methods in Cellular Immunology, W. H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4th ed. Ed. Richard A. Goldsby, Thomas J. Kindt and Barbara A. Osborne, H. Freemand & Co. (2000); Roitt, I., Brostoff, J. and Male D., Immunology 6th ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

### EXAMPLES

The invention will now be described by reference to non-limiting examples.

Mice were inoculated with human ovarian cancer cell lines and allowed to become established (average tumor size of about 100 mm³) prior to treatment. Conjugate dosing is described based on DM1 concentration. Efficacy is reported as both the % of tumor growth for treated vs. control (% T/C) and log cell kill (LCK) determined from the tumor doubling time and the tumor growth delay due to the treatment. Percent T/C values less than or equal to 42% and/or LCK values of 0.5 or greater are considered active; percent T/C values less than 10% are considered highly active (Bissery et al., Cancer Res, 51: 4845-4852 (1991).

### Example 1

### Anti-Tumor Effect of IMGN901 Treatment in OVCAR-3 Human Ovarian Carcinoma Xenografts

The anti-tumor effect of IMGN901 was evaluated in an established subcutaneous xenograft model of ovarian carcinoma. SCID mice were inoculated with OVCAR-3 ovarian carcinoma cells (1×10⁷ cells/animal) injected subcutaneously into the right flank. When the tumors reached about 100 mm³ in size (24 days after tumor cell inoculation), the mice were randomly divided into three groups (6 animals per group). Mice were treated with the single agent IMGN901 at 6.5 mg/kg and 13 mg/kg, respectively, administered intravenously once weekly for three weeks (day 24, 31, 39). A control group of animals received PBS administered intravenously at the same schedule. Tumor growth was monitored by measuring tumor size twice per week. Tumor size was calculated with the formula: length×width×height×½.

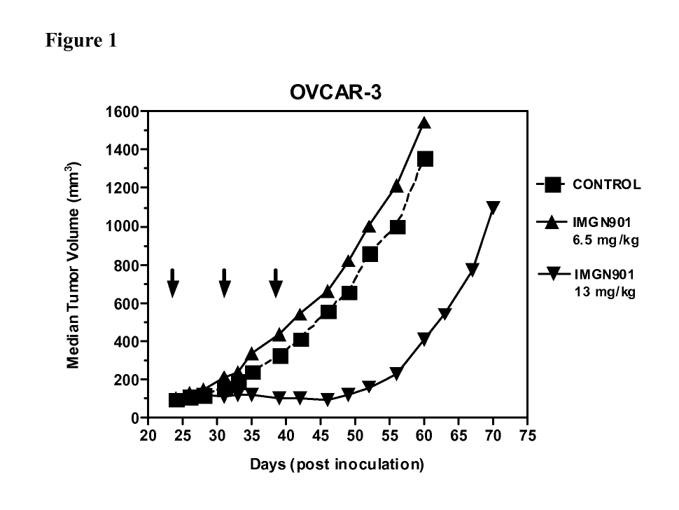

FIG. 1. IMGN901 was active against OVCAR-3 tumors in terms of tumor growth inhibition (T/C=21%) at the 13 mg/kg dose. According to NCI standards the T/C value of 21% is considered to be active. The 6.5 mg/kg dose was inactive.

### Example 2

### Dose-Response Anti-Tumor Activity of IMGN901 Treatment in COLO 720E Human Ovarian Carcinoma Xenografts

The anti-tumor effect of IMGN901 was evaluated in an established subcutaneous xenograft model of ovarian carcinoma. SCID mice were inoculated with COLO 720E ovarian carcinoma cells (1×10⁷ cells/animal) injected subcutaneously into the right flank. (The COLO 720E human ovarian adenocarcinoma cell line was obtained from the European Collection of Cell Cultures (ECACC, catalog no. 93072111).) When the tumors reached about 100 mm³ in size (10 days after tumor cell inoculation), the mice were randomly divided into four groups (6 animals per group). Mice were treated with the single agent IMGN901 at 6, 12 and 24 mg/kg, respectively, administered intravenously once weekly for three weeks (day 10, 17 and 24). A control group of animals received PBS administered intravenously at the same schedule. Tumor growth was monitored by measuring tumor size twice per week. Tumor size was calculated with the formula: length×width×height×½.

Dose-dependent activity of IMGN901 was observed in the COLO 720E xenograft model. IMGN901 was highly active against COLO 720E tumors at the dose of 24 mg/kg once weekly for three weeks. The tumor growth inhibition value (T/C) was 0% which is highly active by NCI standards. All six mice in the group mice exhibited tumor regressions: 6 partial regressions (PR is defined as greater than 50% decrease from initial tumor volume) and 6 complete regressions (CR), with four mice remaining tumor free at the end of the study (119 days). IMGN901 was also active at the dose of 12 mg/kg, weekly for 3 weeks. The tumor growth inhibition value (T/C) was 18%, which is considered active by NCI standards. Four of 6 mice exhibited tumor regressions: 4 partial and 2 complete, with one mouse remaining tumor free at the end of the study. The 6 mg/kg (once weekly for three weeks) dose was inactive.

### Example 3

### Anti-Tumor Effect of Combination Therapy of COLO 720E Human Ovarian Carcinoma Xenografts with IMGN901 and Paclitaxel Plus Carboplatin

The anti-tumor effect of a combination of huN901-DM1 and paclitaxel plus carboplatin was evaluated in an established subcutaneous xenograft model of ovarian cancer. Athymic nude mice were inoculated with COLO 720E human ovarian carcinoma cells (1×10⁷ cells/animal) injected subcutaneously into the right flank. When the tumors reached about 80 mm³ in size (10 days after tumor cell inoculation), the mice were randomly divided into six groups (6 animals per group). Mice were treated with the single agent IMGN901 at a dose of 13 mg/kg once weekly for three weeks (day 10, 17 and 24 post tumor cell inoculation) administered intravenously. Two additional groups of mice were treated with the combination chemotherapy regimen paclitaxel/carboplatin at two dose levels: a high-dose group of paclitaxel (20 mg/kg iv, weekly for 3 weeks)/carboplatin (100 mg/kg ip, single injection) and a low-dose group of paclitaxel (10 mg/kg iv, weekly for 3 weeks)/carboplatin (100 mg/kg ip, single injection). Two additional groups were treated with the combination of IMGN901 and either high-dose or low-dose paclitaxel/carboplatin with the same doses and routes of administration as for individual treatments. Tumor growth was monitored by measuring tumor size twice per week. Tumor size was calculated with the formula: length×width×height×½.

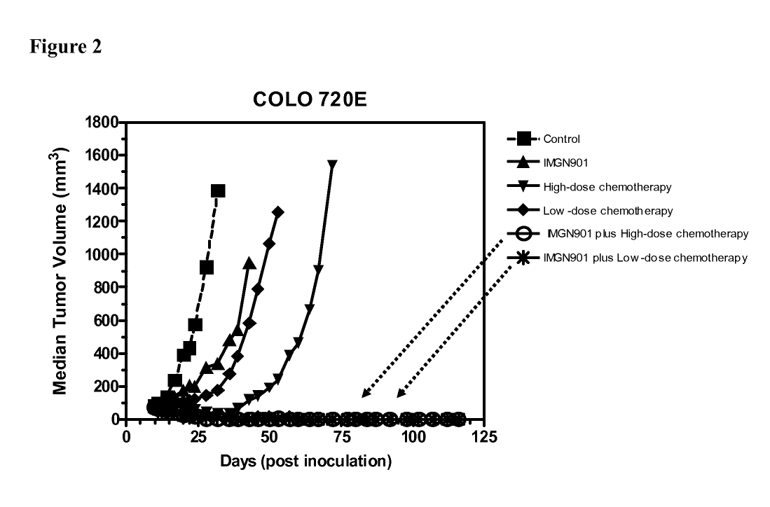

FIG. 2. Single-agent IMGN901 was active against COLO 720E xenografts, with a T/C of 32%, which is considered active by NCI standards. Two of six mice exhibited partial tumor regressions; one of six mice had a complete regression. The chemotherapy treatments were also active; high-dose paclitaxel/carboplatin was highly active (T/C=4%) and PR in 3/6 mice and CR in 2/6 mice and low-dose paclitaxel/carboplatin resulting in a T/C of 15% (active by NCI standards) with no tumor regression observed. Combination of IMGN901 with either high-dose or low-dose paclitaxel/carboplatin chemotherapy was highly active by NCI standards (0% and 1% T/C, respectively) and all mice exhibited complete tumor regressions and remained tumor-free until the end of the study (day 123). There were no tumor-free survivors in either single-agent IMGN901 or chemotherapy alone treatment groups.

### Example 4

### Anti-Tumor Effect of Low-Dose IMGN901 in Combination with Paclitaxel/Carboplatin against COLO 720E Human Ovarian Carcinoma Xenografts

The anti-tumor effect of reduced doses of IMGN901 and paclitaxel plus carboplatin was evaluated in established subcutaneous COLO 720E xenografts. When the tumors reached about 100 mm³ in size (14 days after tumor cell inoculation), the mice were randomly divided into groups of 6 animals each based on tumor volume. Mice were treated with the single agent IMGN901 at a dose of 11.4 mg/kg (qw×3) or the chemotherapeutic combination paclitaxel/carboplatin at either a high-dose (paclitaxel 20 mg/kg, qw×3/carboplatin, 100 mg/kg ip, single injection) or a low-dose (paclitaxel 10 mg/kg, qw×3/carboplatin, 100 mg/kg ip, single injection). Treatment with IMGN901 as a single agent was inactive in this study, with a T/C of 62%. High-dose paclitaxel/carboplatin was highly active with a T/C of 8% whereas the low-dose paclitaxel/carboplatin was inactive resulting in a T/C of 44%. IMGN901 was also evaluated in combination with both high- and low-dose paclitaxel/carboplatin, at the same dose which was tested as a single-agent (11.4 mg/kg, inactive) as well as several lower dose levels (8.5, 5.7, and 2.8 mg/kg) with the same schedules.

FIG. 3. Combinations of IMGN901 at all dose levels with high-dose paclitaxel/carboplatin were highly active. In contrast to only one complete tumor regression (CR) in the chemotherapy alone group (1 of 4 animals), there were CRs in all animals (6 of 6) in the combination groups at IMGN901 dose levels 11.4, 8.5 and 5.7 mg/kg, and CRs in 3 of 6 mice in the lowest dose combination (2.8 mg/kg).

Table 1. Both IMGN901 and low-dose paclitaxel/carboplatin were inactive as single therapies (monotherapies). However, combinations were highly active at IMGN901 dose levels 11.4, 8.5 and 5.7 mg/kg. Although there were no partial (PR) or complete regressions (CR) in the monotherapy groups, dose-dependent tumor regressions were observed in these combination groups. The highest dose combination (IMGN901 at 11.4 mg/kg) resulted in CRs in all animals (6 of 6). Combination at the 8.5 mg/kg dose of IMGN901 resulted in PR in 5 of 6 mice and CR in 3 of 6 mice. The combination of IMGN901 at 5.7 mg/kg, a 50% reduction in IMGN901 from the inactive maximal dose, was highly active with PR in 4 of 5 animals and CR in 3 of % animals. The lowest dose combination (IMGN901 2.8 mg/kg) was inactive.]

TABLE 1

| Treatment | PR | CR | Response |
|---|---|---|---|
| IMGN901- 11.4 mg/kg | 1/6 | 0/6 | inactive |
| Paclitaxel/carboplatin High-dose | 1/4 | 1/4 | highly active |
| IMGN901- 11.4 mg/kg combination | 6/6 | 6/6 | highly active |
| IMGN901- 8.5 mg/kg combination | 6/6 | 6/6 | highly active |
| IMGN901- 5.7 mg/kg combination | 6/6 | 6/6 | highly active |
| IMGN901- 2.8 mg/kg combination | 4/6 | 3/6 | highly active |
| Paclitaxel/carboplatin Low-dose | 0/5 | 0/5 | inactive |
| IMGN901- 11.4 mg/kg combination | 6/6 | 6/6 | highly active |
| IMGN901- 8.5 mg/kg combination | 5/6 | 3/6 | highly active |
| IMGN901- 5.7 mg/kg combination | 4/5 | 3/5 | highly active |
| IMGN901- 2.8 mg/kg combination | 0/6 | 0/6 | inactive |

### Provisos

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted as by a person of ordinary skill in the most closely related art in light of the teachings and guidance.

The breadth and scope of the present invention should not be limited by any of the above-described exemplary embodiments.

What is claimed is:

1. A pharmaceutical composition comprising an antibody or fragment thereof which specifically binds CD56, wherein said antibody or fragment thereof is linked to a cytotoxic compound, wherein said pharmaceutical composition further comprises a taxane compound and a platinum compound, wherein said pharmaceutical composition provides a synergistic effect in the treatment of ovarian cancer.

2. The pharmaceutical composition of claim 1, wherein said cytotoxic compound is an anti-mitotic agent.

3. The pharmaceutical composition of claim 2, wherein said anti-mitotic agent is a maytansinoid.

4. The pharmaceutical composition of claim 3, wherein said maytansinoid is DM1.

5. The pharmaceutical composition of claim 1, wherein said taxane compound is selected from the group consisting of:
(a) paclitaxel;
(b) docetaxel; and
(c) a combination of (a) and (b).

6. The pharmaceutical composition of claim 1, wherein said platinum compound is selected from the group consisting of:
(a) a carboplatin compound;
(b) a cisplatin compound;
(c) an oxaliplatin compound;
(d) an iproplatin compound;
(e) an ormaplatin compound; and
(f) a tetraplatin compound;
(g) any combination of two or more of (a)-(f).

7. The pharmaceutical composition of any of claims 1 to 6, wherein said antibody or fragment thereof is a humanized antibody or a fragment thereof.

8. The pharmaceutical composition of any of claims 1 to 6, wherein the antibody is huN901 or a fragment thereof.

9. The pharmaceutical composition of any of claims 1 to 6, wherein the antibody linked to a cytotoxic compound is IMGN901.

10. A pharmaceutical composition comprising IMGN901, a taxane compound selected from the group consisting of:
(a) paclitaxel;
(b) docetaxel; and
(c) a combination of (a) and (b)
and further comprising a platinum compound is selected from the group consisting of:
(d) a carboplatin compound;
(e) a cisplatin compound;
(f) an oxaliplatin compound;
(g) an iproplatin compound;
(h) an ormaplatin compound; and
(i) a tetraplatin compound;
(j) any combination of two or more of (d)-(i).

11. The pharmaceutical composition of claim 10, wherein said composition comprises IMGN901, paclitaxel and carboplatin.

12. A method of treating ovarian cancer by administration of a therapeutically useful amount of the pharmaceutical composition in any one of claims 1 to 11.

13. The method of claim 12, wherein said administration is to a human.

14. The method of claim 12, wherein said administration is to a non-human mammal.

15. The method of claim 12, wherein the antibody or fragment thereof linked to a cytotoxic compound, the taxane compound, and the platinum compound are administered in a combined dose wherein the individual amount of any one agent or compound in the pharmaceutical composition would be non-therapeutic if administered alone.

16. The method of claim 15, wherein the individual amount of any one agent, compound, antibody or fragment thereof linked to a cytotoxic compound is administered at a non-therapeutic dose to reduce or eliminate toxicity or undesirable side effects.
